(19) United States
(12) Patent Application Publication
(10) Pub. No.: US 2021/0259951 A1
(43) Pub. Date: Aug. 26, 2021

(54) Transmucosal Drug Delivery System

(71) Applicants: John J. Masiz, (US); North Atlantic Holdings LLC, Gloucester, MA (US)

(72) Inventors: John J. Masiz, Topsfield, MA (US); Zhen Zhu, Andover, MA (US)

(73) Assignees: North Atlantic Holdings LLC, Gloucester, MA (US); John J. Masiz, Gloucester, MA (US)

(21) Appl. No.: 17/177,430

(22) Filed: Feb. 17, 2021

Related U.S. Application Data
(60) Provisional application No. 62/979,239, filed on Feb. 20, 2020.

(51) Int. Cl. A61K 9/00; A61K 45/06; A61K 31/522; A61K 31/167; A61K 47/42; A61K 47/44; A61K 47/02; A61K 47/10; A61K 47/26; A61K 47/12; A61K 47/14; A61K 47/22

(52) U.S. Cl. CPC A61K 9/006; A61K 45/06; A61K 31/522; A61K 31/167; A61K 47/42; A61K 47/22; A61K 47/02; A61K 47/10; A61K 47/26; A61K 47/12; A61K 47/14; A61K 47/44

(57) ABSTRACT

The present invention relates to transmucosal delivery systems, methods and kits that include agents to penetrate the mucus, stratified squamous epithelial layer, and basement membrane, to deliver a vasodilatory agent and active agent to the lamina propria and smooth muscle. The formulation of the present invention further includes having a mucolytic agent for thinning or decreasing viscosity of mucus; a solvent; a proteolytic agent that cleaves or fragments long chain proteins to create cellular spacing of the stratified squamous epithelial layer and/or basement membrane, a vasodilatory agent; and at least one active ingredient. The formulation allows for penetration of the active ingredient at the mucosal surface to the smooth muscle. The active ingredient, once at the smooth muscle, is delivered locally to the tissue or systemically to the blood stream.

### RELATED APPLICATION

This application claims the benefit of U.S. Provisional Application No. 62/979,239, entitled, “Transmucosal Drug Delivery System” by John J. Masiz et al., filed Feb. 20, 2020.

The entire teachings of the above application(s) are incorporated herein by reference.

### BACKGROUND OF THE INVENTION

Often drug delivery is administered through the use of pills, injections with needles and/or intravenous delivery. Of these options, pills or oral delivery has been the predominant method for use by patients outside of the doctor's office or hospital. Though convenient for patients to use, oral pills can, in certain cases, inefficiently deliver active ingredients and be far from ideal. Certain problems with oral administration include, for example, gastrointestinal impact, first pass liver effect, and inter-gut and drug-to-drug interactions.

Because of these problems with oral delivery, research has focused on developing alternative delivery methods of active drugs that maintain both the convenience of oral delivery while eliminating the side effects and problems associated with pills and inter-gut delivery. One area of research focus for an alternative drug delivery system has been with transmucosal delivery.

Historically, the success of transmucosal delivery technologies has often been limited. Mucosal delivery systems have included patches, films wafers, emulsions, pastes and sprays. However, these systems have had some success but with only a few drugs. Generally, the few drugs that successfully moved through the mucosal tissue were compounds with a high log-P value, small molecular weight and a linear molecular structure that allowed for passive transmucosal penetration.

Accordingly, a need exists for improved transmucosal delivery systems and methods that provide delivery of a larger variety of pharmaceutical agents and to do so by delivering effective amounts of the active agent with the convenience of traditional pill delivery. A further need exists for an improved transmucosal delivery system that provides more efficient delivery of the active ingredient and to do so systemically or to targeted tissue with a faster onset of action. Yet a further need exists to avoid unwanted effects from oral delivery of medications.

### SUMMARY OF THE INVENTION

The present invention relates to a formulation for transmucosal delivery of an active ingredient to a mammal having a mucosal surface. The mucosal surface includes mucus, a stratified squamous epithelial layer, a basement membrane, a lamina propria and smooth muscle. The inventive formulation includes at least one mucolytic agent for thinning or decreasing viscosity of mucus, at least one solvent, and at least one proteolytic agent that cleaves or fragments long chain proteins to create cellular spacing of the stratified squamous epithelial layer, basement membrane, or a combination thereof, to allow passage of at least one vasodilatory agent and at least one active ingredient. The formulation of the present invention also includes at least one vasodilatory agent and at least one active ingredient. The formulation allows for penetration of the active ingredient at the mucosal surface to the smooth muscle. In an embodiment, the pH of the formulation ranges between about 5.0 and about 6.5. The formulation can further include, for example, a pH regulating agent such as citric acid, hydrochloric acid, potassium hydroxide, sodium hydroxide, sodium carbonate, sodium bicarbonate, or any combination thereof.

The mucolytic agent used in the inventive formulation includes, in an embodiment, acetylcysteine, N-acetylcysteine, L-cysteine, ambroxol, bromhexine, carbocisteine, erdosteine, mecysteine, dornase alfa., althea extract, Marshmallow root, Bromelain, Thyme, Salt Water, Eucalyptol, Rosemary extract, Cineole, Peppermint, Frankincense, Oregano, Bergamot, Nutmeg, Cypress, Camphene, Geranium, Pelargonium sidoides, Cinnamon, Lemon, Citrus, D-limonene (citrus oils) or L-Limonenes (mint oils), Lavender, Lemon grass, Chamomile, Basil and a combination thereof.

The formula of the present invention includes at least one solvent. The solvent can be, for example, at least one nonpolar solvent (e.g., carbon tetrachloride (CCl4), benzene (C6H6), diethyl ether (CH3CH2OCH2CH3), hexane (CH3(CH2)4CH3), methylene chloride (CH2C12), toluene and a combination thereof), at least one polar aprotic solvent (e.g., propylene carbonate, acetone ((CH3)2C═O), ethyl acetate (CH3CO2CH2CH3), dimethyl sulfoxide ((CH3)2SO) (referred to as “DMSO”), acetonitrile (CH3CN), dimethylformamide ((CH3)2NC(O)H), and combination thereof), at least one polar protic solvent (e.g., water (H—OH), methanol, isopropanol, acetic acid (CH3CO—OH) methanol (CH3-OH), ethanol (CH3CH2-OH), n-propanol (CH3CH2CH2-OH), n-butanol (CH3CH2CH2CH2-OH), and a combination thereof), at least one fatty acid (e.g., linoleic acids, linolenic acids, oleic acids, stearic acids, and myristic acids, phospholipids (e.g., phosphatidylcholine, phosphatidylethanolamine, phosphatidylserine, and a combination thereof)), at least one limonene (e.g., D-limonene, L-Limonenes and a combination thereof), or combination thereof.

The proteolytic agent used in the inventive formulation includes, in an embodiment, serine proteases, cysteine proteases threonine proteases, aspartic proteases, glutamic proteases, metalloproteases, asparagine peptide lyases, glucanases or combination thereof. In one aspect, the proteolytic agent includes amyl glucosidase, alpha-amylase, amylase, alpha-glucanase, beta-glucanase, glactomannase, hemicellulase, acid protease, alkaline protease, cellulase I, cellulase II, lipase, lactase, serratio peptidase, exo-oeptidase, endo-peptidase, betaine, maltase, ox bile extract, phytase, pancreatin, pepsin, protease I-IV, pullulanase, sucrase, protease invertase, pectinase, papain, papaya, apple pectin, ginger, tumeric, bromelain, pineapple, peppermint or combination thereof.

The vasodilator of the inventive formulation allows for the active ingredient to be delivered systemically or to local tissue. Examples of vasodilators include amrinone, arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, papaverine, xanthinol nicotinate, diazoxide, hydralazine, minoxidil, and sodium nitroprusside, clonidine, quanaberz, methyl dopa, alpha adrenoceptor, indoramin, phenoxybenzamine, phentolamine, prazosin, PDE-5 inhibitors, sildenafil, tadalafil, adrenergic neuron blocking agents, bedmidine, debrisoquine, guanethidine, ACE inhibitors, benazepril, captopril, cilazapril, enalapril, fosinopril, lisinopril, perindopril, quinapril, ramipril, ganglion blocking agents, pentolinium, trimetaphan, calcium channel blockers, amlodipine, diltiazem, felodipine, isradipine, nicardipine, nifedipine, nimodipine, verapamil, prostaglandins, prostacyclin, thrombuxane A2, leukotrienes, PGA, PGA1, PGA2, PGE1, PGE2, PGD, PGG, PGH, angiotensin II analogs, saralasin, nitroglycerin, labetalol, thrazide, isosorbide dinitrate, pentaerythritol tetranitrate, digitalis, hydralazine, diazoxide, sodium nitroprusside, and a combination thereof.

The active ingredient of the inventive formulation includes any active drug. In an embodiment, the active ingredient is present in the inventive formulation in an amount ranging from about 0.001% w/w and about 30% w/w. Examples of active ingredients include acetaminophen, acetohydoxamic acid, acetophenazine, acyclovir, albuterol, allopurinol, amiloride, amoxicillin, amphetamine, ampicillin, antisense polymers, atenolol, baclofen, beclomethasone, benfotiamine, betamethasone, budesonide, bumetanide, butorphanol, carbamazepine, carphenazine, celacoxhib, cefuroxime, cephradine, chloramphenicol, chlorothiazide, chlorzoxazone, cinoxacin, clorazepate, cloxacillin, cyclacillin, dapsone, dicloxacillin, diethylstilbestrol, dopamine, doxorubicin, erythropoietin, estradiol, fenoprofen, gabapentin, human growth hormone, hydralazine, hydrochlorothiazide, ibuprofen, indomethacin, insulin, isoproterenol, ketoprofen, levodopa, levothyroxine, meclofenamate, melphalan, metformin methyl salicylate, metronidazole, minoxidil, morphine, nadolol, nalidixic acid, naproxen, nomifensine, norfloxacin, oxaprozin, oxycontin, paramethasone, peptide fragments, perphenazine, phenylpropanolamine, pregabalin, probenecid, quinethazone, ritodrine, scopolamine, serotonin, sildenafil, tadalafil, terbutaline, terfenadine, tocainide, terbinafine, triamterene, riamterine, trimethoprim, valacyclovir and any derivatives of these and combinations of the foregoing. The active ingredient can also be a sirtuin inhibitors such as nicotinamide, AIII, coumarin, sirtinol, alpha-NAD, carbamido-NAD, trichostatin A, suramin sodium, apicidin, BML-210, BML-266, depudecin, HC Toxin, ITSA1, nullscript, phenylbutyrate, sodium, scriptaid, splitomicin, or suberoyl bis-hydroxamic acid. Further, the active ingredient can be sirtuin activators such as resveratrol, isonicotinamide, butein, or luteolin. In addition, active ingredients can also be compounds extracted from plants including hemp and cannabis in all of their forms including essential oils, extracts or isolates.

The present invention can further include a transpiration barrier, wherein the transpiration barrier includes at least one of a chemical barrier or a physical barrier.

The present invention includes methods for transmucosal delivery of the inventive formulation described herein. The method includes the steps of applying the formulation to the mucosal surface in one step (e.g., together), in two or more steps (e.g., applying the mucolytic agent to thin the viscosity of the mucus and then the rest of the formulation in one or more steps), or sequentially, wherein the formulation allows for penetration of the active ingredient to at mucosal surface to the smooth muscle. When applying the components of the inventive formulation sequentially, the method includes the steps of administering at least one mucolytic agent for thinning or decreasing viscosity of mucus; administering at least one proteolytic agent that cleaves or fragments long chain proteins to create cellular spacing of the stratified squamous epithelial layer, basement membrane, or a combination thereof, to allow passage of at least one vasodilatory agent and at least one active ingredient in at least one solvent; administering at least one vasodilatory agent; and administering at least one active ingredient. The method can also include, for example, applying an occlusive barrier to the mucosal surface.

The present invention includes systems and kits having the components described herein. In particular, the system and/or kits include at least one mucolytic agent for thinning or decreasing viscosity of mucus; at least one solvent; at least one proteolytic agent that cleaves or fragments long chain proteins to create cellular spacing of the stratified squamous epithelial layer, basement membrane, or a combination thereof, to allow passage of at least one vasodilatory agent and at least one active ingredient; at least one vasodilatory agent; and at least one active ingredient. Kits can also include a set of written instructions for use, by or on said mammal.

Advantageously, the transmucosal delivery formulation of the present invention results in an expanded range of active ingredients that can be delivered through the mucosal membranes. For example, by using formulation of the present invention, higher molecular weight and hydrophilic active agents can be effectively delivered without stomach and first pass liver impact. The inventive formulation addresses the difficult-to-penetrate mucosal layers, including the basement membrane, and addresses the impact that saliva or mucosal fluid has on the active drug. The present invention also allows for the passage of the active drug into general systemic circulation or localized deep organ tissue with a faster and more efficient onset of action of the active ingredients than oral delivery.

### BRIEF DESCRIPTION OF THE DRAWINGS

FIG. 1 is a flow diagram showing the steps of method 100 of the transmucosal delivery system of the present invention.

### DETAILED DESCRIPTION OF THE INVENTION

A description of preferred embodiments of the invention follows.

The present invention overcomes at least three major barriers in order to have a delivery system that successfully and efficiently penetrates the mucosal tissue. The first barrier is the structure of the mucosa itself. As a barrier organ of the body, mucosal tissue consists of various structural layers designed to protect the tissue it is covering. The layers of mucosal tissue, starting from the outside, include the stratified squamous epithelial layer, the basement membrane, the lamina propria, and smooth muscle. In the mouth, the cells of the outer stratified squamous epithelial layer are unkeratinized. This lipophilic layer covers the basement membrane which is a dense protein layer designed to prevent most permeation from foreign objects. In fact, the basement membrane functions to not only prevent foreign particles from penetrating into living tissue, but it also functions to prevent fluids and living tissue constituents from moving out to the stratified squamous epithelial layer. The basement membrane is formed from glycoproteins and is designed to also form the anchoring layer between the epithelial cells and the lamina propria (connective tissue) underlying the basement membrane. The lamina propria, the layer under the basement membrane, is the connective tissue layer which anchors the entire mucosa to the smooth muscle tissue underneath. Taken together, the three structures of the mucosa (epithelial cells, basement membrane and lamina propria), constitute an exceptionally difficult structure for foreign objects, like active drugs, to penetrate.

The second effective barrier that the formulation of the present invention overcomes relates to mucosal secretions from mucosal tissue, which in the case of the mouth, is saliva. Saliva is approximately 99% water but also contains other components like electrolytes, mucus, epithelial cells, enzymes and antimicrobial agents. Specifically, mucin, the principal component in mucus, is a high molecular weight glycosylated protein which is secreted by the epithelial tissue solely as a penetration barrier. While saliva functions as a wetting agent and the commencement of the digestive process, it also effectively acts as a barrier to mucosal tissue penetration. Saliva solubilizes hydrophilic compounds, neutralizes acids and initiates the swallowing process. These functions work to thwart mucosal penetration and limit the potential impact of an active ingredient.

The inventive formulation defeats a third effective barrier to efficient transmucosal penetration and relates to the lack of access to the circulatory system. The epithelial layers contain no blood vessels. Neither does the basement membrane or the lamina propria. Although it is not vascularized, the lamina propria does contain a rich amount of plasma which feeds the epithelial cells by way of diffusion through the basement membrane. This passive indirect plasma flow effectively isolates any mucosal penetrant from any meaningful access to the circulatory system further thwarting an efficient transmucosal drug delivery system.

The present invention creates a solution which can be placed onto the mucosal tissue that will do the following: 1. Decrease the viscosity of and further solubilize into the mucosal secretion (saliva in the mouth); 2. Penetrate through the various mucosal membrane layers; and, 3. efficiently allow the active drug to access the circulatory system located within the smooth muscle tissue below the lamina propria.

In the body, mucosal tissue lines the various cavities and organs with a major example being the lining of the oral cavity. The words “transmucosal” and “mucosal” refer to oral mucosa (including sublingual structures) as well as all mucosal tissue of the nose, esophagus, trachea, lungs, stomach, intestines, and vagina.

In accordance with illustrative embodiments of the present invention, the present invention relates to a transmucosal drug delivery formulation that includes, but not limited to, at least one solvent, at least one mucolytic agent, at least one proteolytic agent, at least one vasodilator, and at least one active agent. The formulation of the present invention can further include a solvent that acts as a carrier vehicle (e.g., one that contains ethyl alcohol, stabilizers, water and various solubilizers) and/or a pH regulating agent, as further described herein. In an embodiment, the pH regulating agent keeps the formulation at a pH between about 5.0 and 6.5 (e.g., 5.0, 5.1, 5.2, 5.3, 5.4, 5.5, 5.6, 5.7, 5.8, 5.9, 6.0, 6.1, 6.2, 6.3, 6.4, 6.5). Without the components of the present invention, the structure of the mucosal tissue, mucosal secretions and lack of circulatory access would prevent efficient transmucosal drug delivery from taking place except for a few highly limited compounds.

In a particular embodiment, the present invention provides a novel transmucosal delivery system that has one or more components described herein in a carrier vehicle, at a pH of between about 5 and about 6.5 to be delivered into the blood stream or deep tissue. The entire solute may contain excipients to provide stability and pH control.

Each component can act (work) by itself, or in combination with other agents/chemicals. In certain embodiments, some components can serve multiple roles. The carrier solution/solvent functions by holding the vasodilator, proteolytic enzyme, mucolytic agent and active drug components into contact with the epithelial cell layer of the mucosa in a form, and at a pH and for a period of time sufficient to allow for the mucolytic agent to initiate penetration. Once initial penetration takes place into the epithelial tissue, the proteolytic enzyme can accelerate penetration deeper into the mucosal tissue allowing the vasodilators to stimulate blood flow sufficient to carry the active components either into general circulation or into deep tissue. The pH regulating agent is designed to control the pH so that it is in a range between about 5 and about 6.5 (slightly acidic) which will provide for optimal penetration into the epithelial cell layers without generating excess saliva. Examples of pH regulating agents include citric acid, hydrochloric acid, potassium hydroxide, sodium hydroxide, sodium carbonate, sodium bicarbonate, and the like.

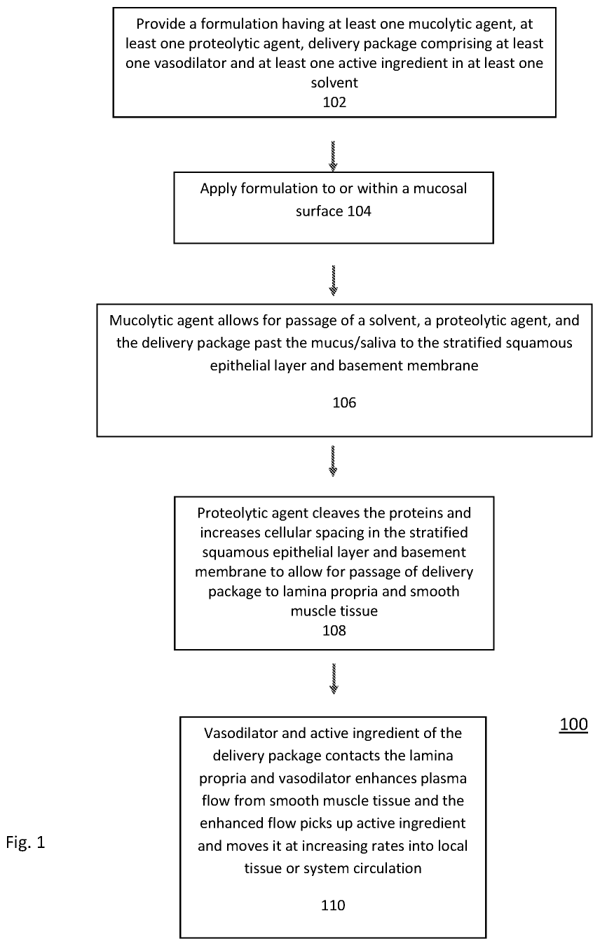

FIG. 1 is a flow diagram, which outlines steps of method 100 for allowing an active agent to penetrate the mucosa to get to the smooth muscle. The inventive formulation enters the mucosal tissue, penetrating through the basement membrane and lamina propria whereby the vasodilators access and enhance localized blood flow which delivers the active agent into the blood stream or into deep tissue. In particular, the steps of method 100 include providing a formulation having at least one mucolytic agent, at least one proteolytic agent, a delivery package comprising at least one vasodilator and at least one active ingredient in at least one solvent in step 102. Step 104 involves applying the formulation to or within the mucosal surface. Such application can be done with or without an applicator device (e.g., aerosol containers, spray containers, dissolving films, pastes, swab applicators and dropper applicators). In Step 106, the mucolytic agent, further described herein, allows for passage of a solvent, a proteolytic agent, and the delivery package past the mucus/saliva to the stratified squamous epithelial layer and basement membrane. Once at the stratified squamous epithelial layer and basement membrane, Step 108 utilizes a proteolytic agent that cleaves the proteins in these layers, and therefore increases cellular spacing. The increased cellular spacing allows for passage of the delivery package to lamina propria and smooth muscle tissue. Once at the lamina propria and smooth muscle, the vasodilator of Step 110 enhances plasma flow from the smooth muscle tissue and the enhanced flow picks up the active ingredient and moves it at increasing rates through these layers into local tissue or system circulation.

Solubilizing (Solvent) Agents

The solvent or solubilizing agents (used interchangeably) refers to one or more chemical compounds that are used to solubilize the remaining ingredients allowing them to become part of the carrier solute that will function as the delivery vehicle. The solvent can optionally also act as a penetration agent which is an agent that can penetrate the stratified epithelial layer. In an embodiment, the solvent used in the inventive transmucosal delivery system can be one or more nonpolar solvents, one or more polar aprotic solvents (including dipolar aprotic solvents), one or more polar protic solvents, a fatty acid, one or more limonenes, or a combination thereof.

Any polar or non-polar solvent may be used either singularly or in combination. In an embodiment, a preferred solvent includes ethanol. The structure of ethanol is such that that it can dissolve polar, hydrophilic and nonpolar hydrophilic compounds. Further, ethanol has a low level of toxicity which is needed for a transmucosal delivery system.

Polar solvents are those that have large dipole moments (aka “partial charges”) and generally contain bonds between atoms with very different electronegativities, such as oxygen and hydrogen. Polarity can be measured using the dielectric constant or from directly measuring the dipole moment. Non-polar solvents generally have bonds between atoms with similar electronegativities, such as carbon and hydrogen. Examples of non-polar solvents encompass carbon tetrachloride (CCl4), benzene (C6H6), diethyl ether (CH3CH2OCH2CH3), hexane (CH3(CH2)4CH3), and methylene chloride (CH2C12). An example of a non-polar, protic solvent further includes toluene.

Furthermore, protic solvents are those that contain a hydrogen atom linked to an oxygen (hydroxyl group) or to a nitrogen (amine group). Protic solvents are able to donate protons (H+). Conversely, aprotic solvents are those that do not donate hydrogen and cannot hydrogen bond with themselves but may accept hydrogen. Polar protic agents have high dielectric constants and high polarity. Examples of polar protic solvents include water (H—OH), methanol, isopropanol, acetic acid (CH3CO—OH), methanol (CH3-OH), ethanol (CH3CH2-OH), n-propanol (CH3CH2CH2-OH), and n-butanol (CH3CH2CH2CH2-OH). Polar aprotic solvents exhibit intermediate dielectric constants, they are polar and are highly miscible in water. An example of polar aprotic solvents is propylene carbonate. Examples of dipolar aprotic solvents include acetone ((CH3)2C═O), ethyl acetate (CH3CO2CH2CH3), dimethyl sulfoxide ((CH3)2SO) (referred to as “DMSO”), acetonitrile (CH3CN), and dimethylformamide ((CH3)2NC(O)H). In an embodiment, polar protic or polar aprotic solvents are preferred.

Additional examples of some solvents include individual fatty acids, fatty acid esters, polyols, amides, various anionic, cationic and nonionic surfactants such as but not limited to sodium laurate and sodium lauryl sulfate, phospholipids, cholesterol and cholesterol derivatives, m-pyrrole, dimethyl acetamide, limonene, sphingo lipids, ceramides, terpenes, alkenones, menthol, various organic acids, such as but not limited to salicylic acid, citric and succinic acid, prostaglandins, dimethyl sulfoxide, decyl-methyl sulfoxide, urea, sulfoxide alcohols, and plant extract oils. Suitable fatty acids include without limitation: linoleic acids, linolenic acids, oleic acids, stearic acids, and myristic acids. Phospholipids include without limitation phosphatidylcholine, phosphatidylethanolamine, and phosphatidylserine. Plant extract oils include, but are not limited to, oils of peanut, grape seed, hemp, borage, olive, sunflower, soy-bean, eucalyptus, monoi and macadamia.

Solvents can also be alcohols such as ethyl alcohol, isopropyl alcohol, and methyl alcohol. Solubilizing agents can also include sugars such as dextrose, or vitamins like vitamin E, C or D.

Limonenes can be used as a solvent in the present invention to penetrate the epithelial layer include D-limonene (citrus oils) or L-Limonenes (mint oils), and optionally together with a liquid hydrocarbon.

Some solubilizing agents are commercially available from Fisher Scientific (Pittsburgh Pa., USA), Spectrum Chemical MFG Corp (New Brunswick, N.J., USA), or BASF (Florham Park N.J., USA). Solubilizing agents now known, or developed in the future, can be used in the compositions and methods of the inventive transmucosal delivery system so long as the agents allow solubilization of the components and can optionally act as a carrier.

In an embodiment, the solubilizing agent can be included in the formulation in sufficient concentration and in effective amounts to allow for solubilization of the components of the transmucosal formulation. In an embodiment, a pharmaceutical transmucosal formulation includes a concentration of solvents from about 1% w/w to about 90% w/w (e.g., 1, 5, 10, 15, 20, 25, 30, 35, 40, 45, 50, 55, 60, 65, 70, 75, 80, 85, 90% w/w).

Proteolytic Agents

The transmucosal delivery system and methods of the present invention utilize one or more proteolytic agents, proteases, or enzymes. A protease (also called a peptidase or proteinase) is an enzyme that catalyzes or increases the rate of proteolysis, the breakdown of proteins into smaller polypeptides or single amino acids. As used herein, proteolytic agents are those that cleave or fragment long chain proteins to create cellular spacing. A proteolytic agent can be used by itself in the inventive formulation, or in combination with others. By using a proteolytic agent in the transmucosal formulation and having the mucolytic agents move them to the epithelial layer and/or basement membrane, the proteins in these layers will be cleaved. In certain embodiments in which the solvent also acts as a penetration agent of epithelial layer, the proteolytic agent need only cleave the proteins of the basement membrane. This protein cleavage causes cellular spacing to thereby allow the vasodilator and active ingredient to move through to the lamina propria and smooth muscle.

Proteolytic agents include proteolytic enzymes or proteases and refer to any enzymes that can cleave or fragmentize long chain proteins to create cellular spacing. There are various classes of proteases that can be included in the formulation of the present invention. Such proteases include Serine Proteases (e.g., Subtilisin, Prolyl oligopeptidase, D-Ala-D-Ala peptidase C, Cytomegalovirus assemblin, Lon-A peptidase, Clp protease, Phage K1F endosialidase CIMCD self-cleaving protein, Nucleoporin 145, Lactoferrin, Murein tetrapeptidase LD-carboxypeptidase, Rhomboid-1, Chymotrypsin A, Penicillin G acylase precursor, Dipeptidase E, DmpA aminopeptidase), Cysteine Proteases (e.g., Papain, bromelain, cathepsin K, calpain, Caspase-1, Adenain, Pyroglutamyl-peptidase I, Sortase A, Hepatitis C virus peptidase 2, Sindbis virus-type nsP2 peptidase, Dipeptidyl-peptidase VI, DeSI-1 peptidase, TEV protease, Amidophosphoribosyltransferase precursor, Gamma-glutamyl hydrolase, Hedgehog protein, DmpA aminopeptidase), Threonine Proteases (e.g., archaean proteasome, beta component, ornithine acetyltransferase), Aspartic Proteases (e.g., BACE1, BACE2, Cathepsin D, Cathepsin E, Chymosin, Napsin-A, Nepenthesin, Pepsin, Presenilin, Renin), Glutamic Proteases (e.g., scytalidoglutamic peptidase (eqolisin) and aspergilloglutamic peptidase), Metalloproteases (e.g., Exopeptidases, metalloexopeptidases), Asparagine Peptide Lyases, and Glucanases. Examples of specific proteolytic enzymes include but are not limited to Amyl glucosidase, Alpha-Amylase, Amylase, Alpha-Glucanase, Beta-Glucanase, Glactomannase, Hemicellulase, Acid Protease, Alkaline Protease, Cellulase I, Cellulase II, Lipase, Lactase, Serratio Peptidase, Exo-Peptidase, Endo-Peptidase, Betaine, Maltase, Ox Bile Extract, Phytase, Pancreatin, Pepsin, Protease I-IV, Pullulanase, Sucrase, Protease Invertase, Pectinase, Papain, Papaya, Apple Pectin, Ginger, Tumeric, Bromelain, Pineapple, and Peppermint.

In an embodiment, the proteolytic agents can be included in the formulation in sufficient concentration (and therefore at sufficient activity) to cleave or fragmentize long chain proteins and to create cellular spacing in the epithelial layer and/or basement membrane. In an embodiment, a pharmaceutical transmucosal formulation includes a concentration of proteolytic agents from about 0.2% w/w to about 10% w/w (e.g., 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.5, 4.0, 4.5, 5.0, 5.5, 6.0, 6.5, 7.0, 7.5, 8.0, 8.5, 9.0, 9.5% w/w) of the formula. Such proteolytic agents are commercially available from Millipore Sigma (St. Louis Mo. USA) or Fisher Scientific (Waltham Mass. USA). Proteolytic agents now known, or developed in the future, can be used in the formulations of the inventive transmucosal delivery system so long as they cleave or fragmentize long chain proteins and create cellular spacing in the epithelial layer and/or basement membrane.

Mucolytic Agents

Mucolytic Agents refers to a class of compounds that decrease the viscosity of mucus. Mucus is a secretion of epithelial tissue that consists of glycosylated proteins. These glycosylated proteins can pose a significant barrier to penetration into the epithelial tissue. Mucolytic agents for the inventive formation are those that thin or substantially diminish the viscosity of the mucus, thereby allowing the remaining components of the inventive formula to have enhanced contact directly with the epithelial tissue. Mucolytic compounds include by example but are not limited to acetylcysteine, N-acetylcysteine, L-cysteine, ambroxol, bromhexine, carbocisteine, erdosteine, mecysteine, dornase alfa, Althea extract, Marshmallow root, Bromelain, Thyme, Salt Water, Eucalyptol, Rosemary extract, Cineole, Peppermint, Frankincense, Oregano, Bergamot, Nutmeg, Cypress, Camphene, Geranium, Pelargonium sidoides, Cinnamon, Lemon, Citrus, D-limonene (citrus oils) or L-Limonenes (mint oils), Lavender, Lemon grass, Chamomile, and Basil.

In an embodiment, the mucolytic agents can be included in the formulation in sufficient concentration (and therefore at sufficient activity) to thin or diminish the viscosity of the mucus. In an embodiment, a pharmaceutical transmucosal formulation includes a concentration of mucolytic agents from about 0.01% w/w to about 10% w/w (e.g., 0.01, 0.05, 0.1, 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.5, 4.0, 4.5, 5.0, 5.5, 6.0, 6.5, 7.0, 7.5, 8.0, 8.5, 9.0, 9.5, 10.0% w/w) of the formula. Such proteolytic agents are commercially available from Millipore Sigma (St. Louis Mo. USA) or Fisher Scientific (Waltham Mass. USA). Mucolytic agents now known, or developed in the future, can be used in the formulations of the inventive transmucosal delivery system so long as they thin or diminish the viscosity of the mucus to allow the rest of the formulation to contact the epithelial layer.

Delivery Package

The delivery package includes the active pharmaceutical agents that are targeted to reach the smooth muscle of the mucosal tissue. The delivery package includes at least two types of compounds: the therapeutic drug or drugs having an “active agent” or “active ingredient” and a vasodialator.

Vasodilatory Agent

A vasodilatory agent or vasodilator refers to circulatory enhancement agent or other agents that are used to increase blood flow and the flow of other fluids below the basement membrane in order to enhance contact between the blood supply in the smooth muscle and the at least one active ingredient. Such vasodilatory agents include vasodilators and pro-drugs of such components.

The vasodilator can be in an amount that is effective to cause dilation of capillaries in the smooth muscle, increase blood in the smooth muscle, and/or increase fluid or plasma flow through from the lamia propria to the smooth muscle. The species of vasodilator can be chosen depending on the speed with which the active drug should move from the lamina propria/smooth muscle and therefore affect the tissue being targeted. Aggressive vasodilators like tolazoline, sodium nitroprusside and papaverine can be used to quickly move the active drug from the lamina propria/smooth muscle into the bloodstream. Mild vasodilators like arginine can be used to slowly release the drug from the lamina propria/smooth muscle into the blood stream so that it can be predominately taken up by the localized tissue or provide release over an extended period of time.

The vasodilator (or mixture of vasodilators) in the formulation can be chosen from the classes of endothelium-dependent vasodilators, endothelium-independent vasodilators and prostaglandin-based vasodilators to elicit the production of endogenous prostaglandins. Prodrugs of any of the foregoing vasodilators can also be used. In an embodiment, inclusion of the vasodilator in the formulation will relax or dilate the arteries and arterioles and therefore increase the volume of flow into the capillary network. This increased volume of blood will subsequently result in an increased trans-capillary flux of water from the vessel into the surrounding tissue.

Vasodilators include, for example, amrinone, arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, sodium nitroprusside, and salts thereof. Centrally acting agents include clonidine, quanaberz, and methyl dopa. Alpha adrenoceptor blocking agents include indoramin, phenoxybenzamine, phentolamine, and prazosin. PDE-5 inhibitors include sildenafil and tadalafil. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion blocking agents include pentolinium and trimetaphan. Calcium channel blockers include amlodipine, diltiazem, felodipine, isradipine, nicardipine, nifedipine, nimodipine, and verapamil. Prostaglandins include prostacyclin, thrombuxane A2, leukotrienes, PGA, PGA1, PGA2, PGE1, PGE2, PGD, PGG, and PGH. Angiotensin II analogs include saralasin. Other suitable vasodilators include nitroglycerin, labetalol, thrazide, isosorbide dinitrate, pentaerythritol tetranitrate, digitalis, hydralazine, diazoxide, and sodium nitroprusside, derivatives of these and combinations of the foregoing. Other examples of vasodilators include nitroglycerine, arginine and some arginine derivatives, acetylcholine, sodium nitroprusside, methyl nicotinate, hexyl nicotinate, arachidonic acid, prostaglandin D2, prostaglandin I2, tolazoline, and papaverine. Arginine is a known substrate for nitric oxide synthase and it is known that nitric oxide can exert a vasodilatory effect.

Such vasodilators are commercially available, for example, from Millipore Sigma (St. Louis Mo. USA), Fisher Scientific (Waltham Mass. USA), or Hawaii-Pharm (Honolulu, Hi. (USA). Vasodilators now known, or developed in the future, can be used in the compositions and methods of the inventive transmucosal delivery system so long as the vasodilators create a fluid dynamic event in the lamina propria and/or smooth muscle to move the active drug into either the blood stream, lymphatic system or deep into local tissue.

In an embodiment, a pharmaceutical transmucosal formulation includes a concentration of vasodilatory agent from about 0.001% w/w to about 15% w/w (e.g., 0.001, 0.005, 0.01, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, or 15% w/w) of the formula.

Active Drugs or Active Chemical Ingredient Components

An Active drug or an “active ingredient” refers to any component of a formulation that provides pharmacological activity or other direct effect in the diagnosis, cure, mitigation, treatment, amelioration, or prevention of a disease, condition or disorder. Further, an active ingredient or active chemical ingredient can include any plant extract, essential oil, isolate, distillate or biologic. Examples of active ingredients that are useful in the topically applied pharmaceutical formulations and methods of the instant invention there may be mentioned: antifungal agents; anti-inflammatory agents, such as non-steroidal anti-inflammatory drugs (NSAIDS) and steroidal anti-inflammatory drugs; antibiotics; antiviral agents; anti-neoplastic agents; astringents; anesthetics; systemic drugs; steroid hormones, such as estradiol and testosterone; cosmetic agents, such as skin moisturizers, protectants, and emollients; nutrients, such as vitamins; and ceramides, and others known to those skilled in the art (e.g., those ingredients listed by the U.S. Food and Drug Agency in “Approved Drug Products with Therapeutic Equivalence Evaluations (Orange Book)”, available at: https://www.fda.gov/drugs/drug-approvals-and-databases/approved-drug-products-therapeutic-equivalence-evaluations-orange-book that are judged suitable by those skilled in the art). In an embodiment, the active ingredient is capable of inducing a desired physiological effect on a targeted mucosa or other tissue other than solely a vasodilatory or vasoconstrictory effect.

Specific examples of active ingredients include acetaminophen, acetohydoxamic acid, acetophenazine, acyclovir, albuterol, allopurinol, amiloride, amoxicillin, amphetamine, ampicillin, antisense polymers, atenolol, baclofen, beclomethasone, benfotiamine, betamethasone, budesonide, bumetanide, butorphanol, caffeine, carbamazepine, carphenazine, celacoxhib, cefuroxime, cephradine, chloramphenicol, chlorothiazide, chlorzoxazone, cinoxacin, clorazepate, cloxacillin, cyclacillin, dapsone, dicloxacillin, diethylstilbestrol, dopamine, doxorubicin, erythropoietin, estradiol, fenoprofen, gabapentin, human growth hormone, hydralazine, hydrochlorothiazide, ibuprofen, indomethacin, insulin, isoproterenol, ketoprofen, levodopa, levothyroxine, lidocaine hydrochloride, meclofenamate, melphalan, metformin methyl salicylate, metronidazole, minoxidil, morphine, nadolol, nalidixic acid, naproxen, nomifensine, norfloxacin, oxaprozin, oxycontin, paramethasone, peptide fragments, perphenazine, phenylpropanolamine, pregabalin, probenecid, quinethazone, ritodrine, scopolamine, serotonin, sildenafil, tadalafil, terbutaline, terfenadine, tocainide, terbinafine, triamterene, riamterine, trimethoprim, valacyclovir and any derivatives of these and combinations of the foregoing. The active ingredient can also be a sirtuin inhibitors such as nicotinamide, AIII, coumarin, sirtinol, alpha-NAD, carbamido-NAD, trichostatin A, suramin sodium, apicidin, BML-210, BML-266, depudecin, HC Toxin, ITSA1, nullscript, phenylbutyrate, sodium, scriptaid, splitomicin, or suberoyl bis-hydroxamic acid. Further, the active ingredient can be sirtuin activators such as resveratrol, isonicotinamide, butein, or luteolin. In addition, active ingredients can also be compounds extracted from plants including hemp and cannabis in all of their forms including essential oils, extracts or isolates. Therapeutic drug or active ingredients now known, or developed in the future, can be used in the compositions and methods of the inventive transmucosal delivery system so long as the therapeutic drug or active ingredients can be delivered to the vasculature of the mucosa or tissue underlying the mucosa.

In some embodiments of the invention, the active ingredient comprises a biological agent. Examples of biological agents include peptides, small proteins and protein fragments; antibody fragments; small nucleic acids and nucleic acid fragments such as aptamers and siRNA; or combinations of these.

In an embodiment, the active ingredient can be included in the inventive formulation in sufficient concentration and in effective amounts to confer the desired effect of the active ingredient. The actual effective amounts of the active agent/ingredient or drug can vary according to the specific composition being utilized, the age, weight and condition of the patient. Dosages for a particular individual patient can be determined by one of ordinary skill in the art using conventional considerations, (e.g. by means of an appropriate, conventional pharmacological protocol). In an aspect, dosing will also depend on the therapeutic effect to be achieved for the disease state. In general, the amount of active ingredient present in the inventive formulation ranges from about 0.001% w/w to about 30% w/w (e.g., 0.001, 0.005, 0.01, 0.05, 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30% w/w).

A transmucosal system can be a formulation having the agents described herein. A “formulation” is a preparation in which various chemical substances are combined with an active ingredient. As used herein, a formulation includes a composition of the invention in the form of a solution, emulsion, ointment, cream, lotion, gel, salve, spray, or the like, for mucosal application or delivery of the drug to a patient. In some embodiments, as appropriate, a formulation is used in conjunction with a delivery system (such as a quick dissolving film) impregnated with or containing a composition suitable for mucosal application. The term “patient” or “individual” refers to any animal, including mammals such as a human, non-human primate, mouse, rat, guinea pig, rabbit, pig, horse or dog.

The mucosal delivery system is administered or applied “directly” to an individual. “Direct” application shall mean application of a formulation to a body's mucosal surfaces such as the mouth, tongue, cheek, buccal pouch, vagina, anus, throat, nose, eyes and ears. For purposes of applying a formula, direct application shall include application to the mucosal tissue, microinjection to the mucosa (such as can be achieved with microneedles), or use of devices such as films, aerosols, liquid, sprays, pastes, patches, sonophoresis, iontophoresis or other permeation-enhancing methods, and subsequent injection to the mucosal or submucosal structures.

In general, the application of the transmucosal formulation of the components, e.g., the components of the solvent, proteolytic enzyme, mucolytic agent, vasodilator and active chemical ingredient, can occur simultaneously or sequentially in time. Thus, the term “co-administration” is used herein to mean that the components of the transmucosal delivery system will be administered at times to achieve delivery of the active ingredient. In an embodiment, the formulation is applied in order to penetrate layers of the mucosal epithelium, starting from the outside layers and going inward. Accordingly, in an embodiment, the mucolytic agents are applied first, then the proteolytic enzymes and subsequently or simultaneously by the vasodilators and active ingredients. In other embodiments, the methods of the present invention are not limited to the sequence in which the compounds are administered, so long as the active chemical agent(s) penetrates through the outer layers of the epithelium to the lamina propria/smooth muscle. A transpiration barrier can also be applied sequentially with respect to the other components one or more times.

In some embodiments, the functions of a mucolytic agent, proteolytic enzyme, vasodilator or active agent can be provided by a single compound, or by two or more compounds. In other words, in an embodiment, a single compound can have more than one function, and have more than one functionality.

In some embodiments the transmucosal delivery formula may optionally be used with a transpiration barrier or an occlusive barrier. A “transpiration barrier” shall mean a component such as a solid patch, a hydrophobic chemical component, or a self-assembling chemical component (including components that form gels) that is capable of preventing the saliva or other mucosal fluids from being washed away from contact with the epithelial tissue. An “occlusive barrier” can be used to apply the formula to the mucosa or other tissue (e.g., application device), to allow for timed release or enhanced delivery (e.g., delivery device). In an embodiment, the occlusive barrier is in the form of a physical patch like material or film or a chemical transpirational barrier (silicone, Vaseline etc.).

The formulation can optionally further include penetration enhancers, agents that improve or boost passage of the active ingredient to the smooth muscle. Examples of penetration enhancers include individual fatty acids, fatty acid esters, polyols, amides, various anionic, cationic and nonionic surfactants such as but not limited to sodium laurate and sodium lauryl sulfate, phospholipids, cholesterol and cholesterol derivatives, m-pyrrole, dimethyl acetamide, limonene, sphingolipids, ceramides, terpenes, alkenones, menthol, and various organic acids, such as but not limited to salicylic acid, citric and succinic acid, prostaglandins, decyl methyl sulfoxide, urea, sulfoxide alcohols, and plant extract oils. Suitable fatty acids include without limitation linoleic acids, linolenic acids, oleic acids, stearic acids, and myristic acids. Phospholipids include without limitation phosphatidylcholine, phosphatidylethanolamine, and phosphatidylserine. Plant extract oils include oils of peanut, hemp, borage, olive, sunflower, soybean, monoi and macadamia. The plant extract oil can be mixed with an alcohol such as ethyl alcohol, isopropyl alcohol, and methyl alcohol.

The formulation can also include excipients or carriers such as Stearyl Alcohol, Polysorbate 20, Caprylic/Capric Glyceride, Petrolatum, Beeswax, Lecithin, Dimethicone, Alkylmethyl Siloxane, Stearic Acid, Palmitic Acid, Lanolin, Water Soluble Gums, Linoleic Acid, Isopropyl Myristate, Stearyl Octanoate and Cetyl Octanoate, and Polysorbate 80.

In addition, the present invention can be used with or without a device for the convenient application onto the mucosal tissue. Examples of various devices include, but are not limited to aerosol containers, spray containers, dissolving films, pastes swab applicators and dropper applicators.

Embodiments of the invention can be useful for medical conditions, diseases or disorders such as musculoskeletal diseases, vascular diseases, neurological diseases, viral, bacterial or parasitic diseases, blood disorders, skin diseases, autoimmune diseases, organ diseases, pain, cosmetic purposes and others. The number of diseases is numerous, but some examples include basal cell carcinomas, melanoma, cervical carcinomas, cervical condylomas, genital warts, herpetic lesions, diabetic neuropathy, chemotherapy-derived neuropathy, general neuropathy, benign prostatic hypertrophy, solid tumors, psoriasis, and eczema. In some embodiments, the active ingredient is a sirtuin inhibitor or sirtuin activator and the formulation is applied to the skin of a patient to treat one of these medical conditions. Where accessible, the formulation can be applied to a region of the skin or tissue associated with the medical condition.

A formulation can be tested for its ability to increase circulation using laser Doppler velocimetry measurements. Such measurements are known in the art (see, e.g., Holloway G A Jr, Watkins D W., 1977, Laser Doppler measurement of cutaneous blood flow. J Invest Dermatol., September; 69(3):306-9). The test can be performed on participants after a 20-minute acclimatization period in a warm environment (room temperature 24° C.). For each subject, the blood flow response is measured with the non-invasive test before and after the application of the test formulation and at various intervals of time after the application until the blood flow has returned to a pre-application level. The measurement of skin blood flow can be evaluated using a Laser Doppler Perfusion Imager (LDPI Lisca 2.0, Lisca development AB, Linkoping, Sweden). This apparatus employs a 1 mW Helium-Neon laser beam of 633 nm wavelength, which sequentially scans the tested area. Typically, the maximum number of measured spots is 4096 and the apparatus produces a color-coded image of the tissue perfusion distribution on a computer monitor. The data acquired from the instrument can be statistically analyzed with The Minitab statistical package (Minitab, State College, Pa.) for personal computers. For intra-group comparisons, the paired t-test can be used to compare changes between baseline and the maximal vasodilation. The test can be used for comparison between the two groups of patients. Changes in the microvascular blood flow can be expressed as the difference between the peak response and the baseline blood flow (e.g., in ml/min, laser-doppler velocimetry voltage readout, or other suitable units).

In some embodiments of the invention, application of the formulation can cause an increase in blood flow at or near the region of application. The increase can range from about 1% to greater than about 500%.

Animal models can be used to evaluate the effectiveness of a topically applied formulation in penetrating the skin tissue for either intramucosal or transmucosal systemic distribution of the active ingredient. Animal models that are preferred include pigs, guinea pigs, rabbit and mini-pigs. An example of the procedure used for such a study using guinea pigs is as follows: Male Hartley guinea pigs (250-300 g) are shaved on the back, and an area of 4×4 cm depilated with Nair® depilatory cream. After approximately 24 hours, 0.5 g of test compound in a topical formulation is applied to the 4×4 cm area and covered with an occlusive wrap. At 1, 2, 4, 8 and 24 hours after application, groups of >5 animals are anesthetized with isoflurane, the application area is swabbed with alcohol, blood is removed by cardiac stick, and the skin tissue of the application area is excised. One group of animals is anesthetized, and blood and skin tissue are removed as vehicle control. Blood samples are processed to serum and analyzed for the presence of an active ingredient via HPLC. The skin below the area of compound application on each animal group is excised, weighed, homogenized in a mixture of acetonitrile and 0.1N HCl (50:50 v/v), centrifuged, and the extract analyzed for the presence of active ingredient via HPLC. The amount of active ingredient in the blood and the amount of active ingredient in the skin tissue may be compared to give information about the pharmacokinetics of the active ingredient. For delivery to local tissue, a higher amount in the skin relative to the blood is more efficacious, whereas when the goal is systemic delivery of the active ingredient, a higher distribution in the blood is more efficacious.

The transmucosal delivery system of the present invention is effective in delivering the active ingredient. In some embodiments, the inventive transmucosal delivery system is as effective in delivering the active ingredient, as compared to traditional methods of administration such as oral administration. In the case of certain embodiments, the inventive transmucosal delivery system delivers the active ingredient more effectively, as compared to its oral administration or other administrations (at least about 1, 5, 10, 15, 20, 25, 30, 35, 40, 45, 50, 55, 60, 65, 70, 75, 80, 85, 90, 95, or 100% more effective).

In another embodiment, a kit or system is used for mucosal delivery of an active ingredient to a patient. The kit includes one or more of the following: solute containing a mucolytic agent, a solute containing a proteolytic agent, a solute containing a vasodilator and a solute containing an active ingredient. The kit can also include a set of written instructions for use thereof according to one of the methods of topical delivery described herein. The kit optionally includes an occlusive barrier.

### EXEMPLIFICATION

### Example 1

Part A

### Example 1

Formulation I. the following components were mixed together in the amounts indicated:

| | Amount % | |
|---|---|---|
| Component | by weight | Type |
| Water | 83.795 | |
| Bromelain | 0.6 | Proteolytic Agent |
| Tween 80 | 4.0 | Surfactant/Solubilizer |
| Alcohol SD-40 | 10.0 | Solvent |
| Oleic Acid | 0.1 | Solubilizer |
| Grapefruit Oil | 0.1 | Flavor |
| Peppermint Oil | 0.1 | Mucolytic Agent |
| Papaverine HCl | 0.005 | Vasodilator |
| Caffeine | 1.0 | Active Pharmaceutical |
| | | Ingredient |
| Methylparaben | 0.2 | Preservative |
| Potassium Hydroxide 10% | 0.1 | Neutralizer |
| Sol. | | |

### Example 2

Formulation II. the following components were mixed together in the amounts indicated:

| | Amount % | |
|---|---|---|
| Component | by weight | Type |
| Water | 82.225 | |
| Poloxamer 407 | 1.0 | Surfactant |
| Bromelain | 1.0 | Proteolytic Agent |
| Cremophor RH 40 | 4.0 | Surfactant/Solubilizer |
| Alcohol SD-40 | 10.0 | Solvent |
| Oleic Acid | 0.1 | Solubilizer |
| Grapefruit Oil | 0.1 | Flavor |
| Peppermint Oil | 0.1 | Mucolytic Agent |
| Papaverine HCl | 0.005 | Vasodilator |
| Lidocaine Hydrochloride | 1.0 | Active Pharmaceutical |
| | | Ingredient |
| Methylparaben | 0.2 | Preservative |
| Potassium Hydroxide 10% | 0.27 | Neutralizer |
| Sol | | |

The terms about, approximately, substantially, and their equivalents may be understood to include their ordinary or customary meaning. In addition, if not defined throughout the specification for the specific usage, these terms can be generally understood to represent values about but not equal to a specified value. For example, 1%, 0.9%, 0.8%, 0.7%, 0.6%, 0.5%, 0.4%, 0.3%, 0.2%, 0.1%, or 0.09% of a specified value.

The terms, “comprise,” “include,” “having” and/or plural forms of each are open ended and include the listed items and can include additional items that are not listed. The phrase “And/or” is open ended and includes one or more of the listed items and combinations of the listed items.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1) A formulation for transmucosal delivery of an active ingredient to a mammal, said mammal having a mucosal surface that comprises mucus, a stratified squamous epithelial layer, a basement membrane, a lamina propria and smooth muscle; the formulation comprises:
a) at least one mucolytic agent for thinning or decreasing viscosity of mucus;
b) at least one solvent;
c) at least one proteolytic agent that cleaves or fragments long chain proteins to create cellular spacing of the stratified squamous epithelial layer, basement membrane, or a combination thereof, to allow passage of at least one vasodilatory agent and at least one active ingredient;
d) at least one vasodilatory agent; and
e) at least one active ingredient;
wherein the formulation allows for penetration of the active ingredient at the mucosal surface to the smooth muscle.

2) The formulation of claim 1, wherein the pH of the formulation ranges between about 5.0 and about 6.5.

3) The formulation of claim 2, further comprising a pH regulating agent comprising citric acid, hydrochloric acid, potassium hydroxide, sodium hydroxide, sodium carbonate, sodium bicarbonate, or any combination thereof.

4) The formulation of claim 1, wherein the at least one mucolytic agent comprises acetylcysteine, N-acetylcysteine, L-cysteine, ambroxol, bromhexine, carbocisteine, erdosteine, mecysteine, dornase alfa., althea extract, Marshmallow root, Bromelain, Thyme, Salt Water, Eucalyptol, Rosemary extract, Cineole, Peppermint, Frankincense, Oregano, Bergamot, Nutmeg, Cypress, Camphene, Geranium, Pelargonium sidoides, Cinnamon, Lemon, Citrus, D-limonene (citrus oils) or L-Limonenes (mint oils), Lavender, Lemon grass, Chamomile, Basil and a combination thereof.

5) The formulation of claim 1, wherein the at least one solvent comprises at least one nonpolar solvent, at least one polar aprotic solvent, at least one polar protic solvent, at least one fatty acid, at least one limonene, or combination thereof.

6) The formulation of claim 5, wherein the at least one nonpolar solvent comprises carbon tetrachloride (CCl4), benzene (C6H6), diethyl ether (CH3CH2OCH2CH3), hexane (CH3(CH2)4CH3), methylene chloride (CH2C12), toluene and a combination thereof

7) The formulation of claim 5, wherein the at least one polar aprotic solvent comprises propylene carbonate, acetone ((CH3)2C═O), ethyl acetate (CH3CO2CH2CH3), dimethyl sulfoxide ((CH3)2SO) (“DMSO”), acetonitrile (CH3CN), dimethylformamide ((CH3)2NC(O)H), and combination thereof.

8) The formulation of claim 5, wherein the at least one polar protic solvent comprises water (H—OH), methanol, isopropanol, acetic acid (CH3CO—OH) methanol (CH3-OH), ethanol (CH3CH2-OH), n-propanol (CH3CH2CH2-OH), n-butanol (CH3CH2CH2CH2-OH), and a combination thereof.

9) The formulation of claim 5, wherein the at least one fatty acid comprises linoleic acids, linolenic acids, oleic acids, stearic acids, myristic acids, phosphatidylcholine, phosphatidylethanolamine, phosphatidylserine, and a combination thereof.

10) The formulation of claim 5, wherein the at least one limonene comprises D-limonene, L-Limonenes and a combination thereof.

11) The formulation of claim 1, wherein the at least one proteolytic agent comprises serine proteases, cysteine proteases threonine proteases, aspartic proteases, glutamic proteases, metalloproteases, asparagine peptide lyases, glucanases, or a combination thereof.

12) The formulation of claim 1, wherein the at least one proteolytic agent comprises amyl glucosidase, alpha-amylase, amylase, alpha-glucanase, beta-glucanase, glactomannase, hemicellulase, acid protease, alkaline protease, cellulase I, cellulase II, lipase, lactase, serratio peptidase, exo-oeptidase, endo-peptidase, betaine, maltase, ox bile extract, phytase, pancreatin, pepsin, protease I-IV, pullulanase, sucrase, protease invertase, pectinase, papain, papaya, apple pectin, ginger, tumeric, bromelain, pineapple, peppermint, or a combination thereof.

13) The formulation of claim 1, wherein the vasodilator allows for the active ingredient to be delivered systemically or to local tissue.

14) The formulation of claim 1, wherein the vasodilator comprises amrinone, arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, papaverine, xanthinol nicotinate, diazoxide, hydralazine, minoxidil, and sodium nitroprusside, clonidine, quanaberz, methyl dopa, alpha adrenoceptor, indoramin, phenoxybenzamine, phentolamine, prazosin, PDE-5 inhibitors, sildenafil, tadalafil, adrenergic neuron blocking agents, bedmidine, debrisoquine, guanethidine, ACE inhibitors, benazepril, captopril, cilazapril, enalapril, fosinopril, lisinopril, perindopril, quinapril, ramipril, ganglion blocking agents, pentolinium, trimetaphan, calcium channel blockers, amlodipine, diltiazem, felodipine, isradipine, nicardipine, nifedipine, nimodipine, verapamil, prostaglandins, prostacyclin, thrombuxane A2, leukotrienes, PGA, PGA1, PGA2, PGE1, PGE2, PGD, PGG, PGH, angiotensin II analogs, saralasin, nitroglycerin, labetalol, thrazide, isosorbide dinitrate, pentaerythritol tetranitrate, digitalis, hydralazine, diazoxide, sodium nitroprusside, and a combination thereof.

15) The formulation of claim 1, wherein the active ingredient is present in an amount ranging from about 0.001% w/w and about 30% w/w.

16) The formulation of claim 1, wherein the active ingredient is selected from the group consisting of: acetaminophen, acetohydoxamic acid, acetophenazine, acyclovir, albuterol, allopurinol, amiloride, amoxicillin, amphetamine, ampicillin, antisense polymers, atenolol, baclofen, beclomethasone, benfotiamine, betamethasone, budesonide, bumetanide, butorphanol, carbamazepine, carphenazine, celacoxhib, cefuroxime, cephradine, chloramphenicol, chlorothiazide, chlorzoxazone, cinoxacin, clorazepate, cloxacillin, cyclacillin, dapsone, dicloxacillin, diethylstilbestrol, dopamine, doxorubicin, erythropoietin, estradiol, fenoprofen, gabapentin, human growth hormone, hydralazine, hydrochlorothiazide, ibuprofen, indomethacin, insulin, isoproterenol, ketoprofen, levodopa, levothyroxine, meclofenamate, melphalan, metformin methyl salicylate, metronidazole, minoxidil, morphine, nadolol, nalidixic acid, naproxen, nomifensine, norfloxacin, oxaprozin, oxycontin, paramethasone, peptide fragments, perphenazine, phenylpropanolamine, pregabalin, probenecid, quinethazone, ritodrine, scopolamine, serotonin, sildenafil, tadalafil, terbutaline, terfenadine, tocainide, terbinafine, triamterene, riamterine, trimethoprim, valacyclovir, sirtuin inhibitors, nicotinamide, AIII, coumarin, sirtinol, alpha-NAD, carbamido-NAD, trichostatin A, suramin sodium, apicidin, BML-210, BML-266, depudecin, HC Toxin, ITSA1, nullscript, phenylbutyrate, sodium, scriptaid, splitomicin, or suberoyl bis-hydroxamic acid, sirtuin activators, resveratrol, isonicotinamide, butein, or luteolin, plants extracts, hemp, nicotine, hemp derived compounds, terpenes, and a combination thereof.

17) The formulation of claim 1, further comprising a transpiration barrier, wherein the transpiration barrier includes at least one of a chemical barrier or a physical barrier.

18) A method for transmucosal delivery of a formulation having an active ingredient to a mammal, said mammal having a mucosal surface that comprises mucus, a stratified squamous epithelial layer, a basement membrane, a lamina propria and smooth muscle; the method comprises:
a) applying the formulation to the mucosal surface, wherein formulation comprises:
i) at least one mucolytic agent for thinning or decreasing viscosity of mucus;
ii) at least one solvent;
iii) at least one proteolytic agent that cleaves or fragments long chain proteins to create cellular spacing of the stratified squamous epithelial layer, basement membrane, or a combination thereof, to allow passage of at least one vasodilatory agent and at least one active ingredient;
iv) at least one vasodilatory agent; and
v) at least one active ingredient;
wherein the formulation allows for penetration of the active ingredient at mucosal surface to the smooth muscle.

19) The method of claim 18, wherein the at least one solvent comprises at least one nonpolar solvent, at least one polar aprotic solvent, at least one polar protic solvent, at least one fatty acid, at least one limonene, or combination thereof

20) A method for transmucosal delivery of a formulation having an active ingredient to a mammal, said mammal having a mucosal surface that comprises mucus, a stratified squamous epithelial layer, a basement membrane, a lamina propria and smooth muscle; the method comprises:
a) administering at least one mucolytic agent for thinning or decreasing viscosity of mucus;
b) administering at least one proteolytic agent that cleaves or fragments long chain proteins to create cellular spacing of the stratified squamous epithelial layer, basement membrane, or a combination thereof, to allow passage of at least one vasodilatory agent and at least one active ingredient in at least one solvent;
c) administering at least one vasodilatory agent; and
d) administering at least one active ingredient;
wherein the formulation allows for penetration of the active ingredient at mucosal surface to the smooth muscle.

21) The method of claim 20, wherein the mucolytic agent, proteolytic agent, vasodilatory agent, and active ingredient are applied sequentially.

22) The method of claim 20, wherein the mucolytic agent, proteolytic agent, vasodilatory agent, and active ingredient are applied together.

23) The method of claim 20, further comprising applying an occlusive barrier to the mucosal surface.

24) The method of claim 20, wherein the at least one solvent comprises at least one nonpolar solvent, at least one polar aprotic solvent, at least one polar protic solvent, at least one fatty acid, at least one limonene, or combination thereof.

25) A kit for transmucosal delivery of an active ingredient to a mammal, said mammal having a mucosal surface that comprises mucus, a stratified squamous epithelial layer, a basement membrane, a lamina propria and smooth muscle; the formulation comprises:
a) at least one mucolytic agent for thinning or decreasing viscosity of mucus;
b) at least one solvent;
c) at least one proteolytic agent that cleaves or fragments long chain proteins to create cellular spacing of the stratified squamous epithelial layer, basement membrane, or a combination thereof, to allow passage of at least one vasodilatory agent and at least one active ingredient;
d) at least one vasodilatory agent; and
e) at least one active ingredient;
wherein the kit creates a formulation that allows for penetration of the active ingredient at the mucosal surface to the smooth muscle.

26) The kit of claim 25, further comprising a set of written instructions for use, by or on said mammal.

27) The kit of claim 25, wherein the at least one solvent comprises at least one nonpolar solvent, at least one polar aprotic solvent, at least one polar protic solvent, at least one fatty acid, at least one limonene, or combination thereof.
